(19) United States
(12) Patent Application Publication
(10) Pub. No.: US 2024/0008456 A1
(43) Pub. Date: Jan. 11, 2024

(54) WEARABLE DEVICE FOR PET

(71) Applicants: MAX.K KOREA Inc., Seoul (KR); Max K LLC, Houston, TX (US)

(72) Inventors: Kibun Max KOO, Houston, TX (US); Sang Min AN, Seoul (KR)

(73) Assignees: MAX.K KOREA Inc., Seoul (KR); Max K LLC, Houston, TX (US)

(21) Appl. No.: 18/183,421

(22) Filed: Mar. 14, 2023

(30) Foreign Application Priority Data
Jul. 5, 2022 (KR) 10-2022-0082309

(51) Int. Cl. A01K 29/00

(52) U.S. Cl. CPC A01K 29/005

(57) ABSTRACT

The present disclosure relates to a wearable device for a pet and, more particularly, to a wearable device for the pet that is worn on body parts of a pet to collect information, including biometric information, movement information, surrounding environment information, or a combination thereof, detected from not only the pet but also surroundings of the pet. The device allows the collected detection information to be registered with a pet management server, and allows an authorized user to access the pet management server and view the detection information for the pet to extensively monitor health status and surroundings of the pet.

### CROSS REFERENCE TO RELATED APPLICATION

This application claims priority to Korea Patent Application No. 10-2022-0082309 filed on Jul. 5, 2022, the content of which is expressly incorporated by reference in its entirety.

### TECHNICAL FIELD

The present disclosure relates to a wearable device for a pet and, more particularly, to a wearable device for the pet that is worn on body parts of a pet to collect information, including biometric information, movement information, surrounding environment information, or a combination thereof, detected from not only the pet but also surroundings of the pet. The device allows the collected detection information to be registered with a pet management server, and allows an authorized user to access the pet management server and view the detection information for the pet to extensively monitor health status and surroundings of the pet.

### BACKGROUND

A companion animal (hereinafter referred to as “pet”) refers to animals, including dogs and cats, that people like, keep close to them, and view as family members. In an aging and individualized modern society where families are becoming smaller, the role of a pet is expanding to a being that provides companionship and shares feelings and emotions with family members.

Against this backdrop, a variety of products and systems for pet health management are being developed, and with the increase in pets, interest in these products and systems is increasing day by day, and thus technology development is still actively underway.

Indeed, dealing with pets is a matter of great care management. However, most pet owners have adopted only simple methods such as having an automatic feeder to discharge pet food for a pet left alone when a predetermined time has passed after their owner goes out, or showing a video of their owner to the pet. When simply providing feed or playing video repeatedly in this way, the pet responds at first, but after habituation, the pet does not react much, which makes it difficult to achieve pet management effects.

Moreover, with the existing management methods using the automatic feeder or video, there is no way for the owner to check health status of their pet, and biometric information, movement information, location information, and surrounding environment information of the pet may not be ascertained as desired. In addition, there are times when the owner cannot stay with their pet due to going to work or going out, and in these times pet may show abnormal behavior triggered by anxiety and stress. Thus, it is necessary to understand the behavior or condition of the pet left alone.

Korean Patent No. 2380481 (2022 Apr. 1) describes a method for analyzing various behaviors of a cat using an accelerometer mounted around a neck of the cat.

Korean Patent No. 2241712 describes a system and method for effectively managing pets or companion animals based on the detection of health conditions of pets using the IoT method and analysis of behavior patterns.

There is a need and a desire for improved pet wearables.

### BRIEF SUMMARY OF THE EMBODIMENTS

According to embodiments, a wearable device is provided including a biometrics measurement unit configured to measure biometrics of a pet; a movement measurement unit configured to measure movements of the pet; and a relevant information collection unit configured to collect relevant information related to the pet from peripheral devices near the pet, wherein the relevant information collection unit is configured to expand and collect the relevant information including biometric information, movement information, surrounding environment information, or a combination thereof generated as the pet approaches or uses the peripheral devices.

In addition, the wearable device for a pet further includes a detection information transmission unit configured to transmit detection information including biometric information, movement information, and collection information, or a combination thereof measured and collected to a pet management server, and transmits the detection information to the pet management server for registration through low-power wide-area networks including LoRa, LTE cat, WiFi or NB-IoT via repeaters including base stations or access points, so that a user connects to the pet management server with his/her user terminal and monitor behavior or condition of the pet.

In addition, the wearable device for a pet further includes a peripheral device access unit configured to perform a communication connection with the peripheral devices, and receives detection information, including biometric information, movement information, surrounding environment information, detected by the peripheral devices, wherein the peripheral device access unit receives the detection information detected by the peripheral devices through NFC, BLE, Bluetooth, etc., and the peripheral devices includes a pet house, tableware, toys, medical measuring devices and instruments, medical equipment.

In addition, the biometrics measurement unit measures the biometrics of the pet by using infrared sensors, and the wearable device for a pet determines an average value or a median value of measurement values measured by each of the infrared sensors as the biometrics of the pet, and corrects the biometrics of the pet by reflecting external temperature change.

In addition, the wearable device for a pet further includes a location checking unit configured to check location information on the pet, and transmits the location information checked to the pet management server for registration so that an exercise record of a pet is checked by tracking a movement path of the pet, or a pet loss situation is responded.

In addition, the wearable device for a pet further includes a vibration generator configured to generate vibration according to a control signal received from the pet management server, and a sound generator configured to output a preset sound or a recorded voice of a user according to a control signal received from the pet management server.

In addition, the wearable device for a pet transmits detection information including biometric information, movement information, and collection information, or a combination thereof to a pet management server by using a non-subscribed user terminal that is not subscribed to a paid communication service or a subscribed user terminal that is subscribed to a paid communication service as a repeater, wherein the non-subscribed user terminal receives the detection information from the wearable device for a pet through NFC, BLE, Bluetooth, etc. and transmits the detection information received to the pet management server only through WiFi, while the subscribed user terminal receives the detection information from the wearable device for a pet through NFC, BLE, Bluetooth, etc. and transmits the detection information received to the pet management server through broadband wired/wireless networks such as 5G, WiFi, wired/wireless Ethernet, CDMA, LTE, etc.

In addition, according to an embodiment of the present disclosure, there is provided a method for operating a wearable device for a pet. The method includes: measuring, by the wearable device for a pet, biometrics of a pet; measuring, by the wearable device for a pet, movements of the pet; and collecting relevant information related to the pet from peripheral devices near the pet, wherein the collecting relevant information is configured to expand and collect the relevant information including biometric information, movement information, surrounding environment information, or a combination thereof generated as the pet approaches or uses the peripheral devices.

In addition, the method for operating a wearable device for a pet further includes transmitting, by the wearable device for a pet, detection information measured and collected to a pet management server, and allows transmission of the detection information to the pet management server for registration through low-power wide-area networks including LoRa, LTE cat, WiFi or NB-IoT via repeaters including base stations or access points, so that a user connects to the pet management server with his/her user terminal and monitor behavior or condition of the pet.

In addition, the method for operating a wearable device for a pet further includes contacting, by the wearable device for a pet, peripheral devices near the pet to perform a communication connection with the peripheral devices and receives detection information, including biometric information, movement information, surrounding environment information, detected by the peripheral devices, wherein the wearable device for a pet receives the detection information detected by the peripheral devices from the peripheral devices through NFC, BLE, Bluetooth, etc., and the peripheral devices includes a pet house, tableware, toys, medical measuring devices and instruments, medical equipment.

In addition, the method for operating a wearable device for a pet further includes determining an average value or a median value of measurement values measured by each of the infrared sensors as the biometrics of the pet, and correcting the biometrics of the pet by reflecting external temperature change.

In addition, the method for operating a wearable device for a pet further includes checking, by the wearable device for a pet, location information on the pet, and allows transmission of the location information checked to a pet management server for registration so that an exercise record of a pet is checked by tracking a movement path of the pet, or a pet loss situation is responded.

In addition, the method for operating a wearable device for a pet further includes vibration generating, by the wearable device for a pet, to generate vibration according to a control signal received from the pet management server, and sound generating to output a preset sound or a recorded voice of a user according to a control signal received from the pet management server.

In addition, the method for operating a wearable device for a pet further includes transmitting, by the wearable device for a pet, detection information including biometric information, movement information, and collection information, or a combination thereof to a pet management server by using a non-subscribed user terminal that is not subscribed to a paid communication service or a subscribed user terminal that is subscribed to a paid communication service as a repeater, wherein the non-subscribed user terminal receives the detection information from the wearable device for a pet through NFC, BLE, Bluetooth, etc. and transmits the detection information received to the pet management server only through WiFi, while the subscribed user terminal receives the detection information from the wearable device for a pet through NFC, BLE, Bluetooth, etc. and transmits the detection information received to the pet management server through broadband wired/wireless networks such as 5G, WiFi, wired/wireless Ethernet, CDMA, LTE, etc.

As described above, a wearable device for a pet of the present disclosure is worn on body parts of a pet to collect detection information periodically measured directly from the pet including biometric information, movement information, surrounding environment information, or a combination thereof and receive detection information generated when the pet uses any of the peripheral devices provided in a fixed or flexible form in a predetermined space (a veterinary hospital, dog cafe, home, office, etc.) from the corresponding peripheral device through near field communication (NFC), Bluetooth low energy (BLE), Bluetooth, etc., and register the collected detection information with a pet management server, so that an owner of the pet (including family members) can freely access the pet management server anytime, anywhere through his/her user terminal and browse the time-sequentially stored information about their pet, enabling extensive monitoring health status and surroundings of the pet.

Other aspects, features, and techniques will be apparent to one skilled in the relevant art in view of the following detailed description of the embodiments.

### BRIEF DESCRIPTION OF THE DRAWINGS

The features, objects, and advantages of the present disclosure will become more apparent from the detailed description set forth below when taken in conjunction with the drawings in which like reference characters identify correspondingly throughout and wherein:

FIG. 1 is a view showing the overall configuration for the processing of detection information related to the health status and behavior of a pet, including a wearable device for a pet according to an embodiment of the present disclosure.

FIG. 2 is a block diagram showing the configuration of the wearable device for a pet according to the embodiment of the present disclosure in more detail.

FIG. 3 is a view showing the process of transmitting the detection information received from peripheral devices to the pet management server together with the detection information directly detected from the pet in the wearable device for a pet according to the embodiment of the present disclosure.

FIG. 4 shows views of other transmission examples of the detection information used in the wearable device for a pet according to the embodiment of the present disclosure.

FIG. 5 is a flowchart showing in detail the operation process of a method for operating the wearable device for a pet according to the embodiment of the present disclosure.

### DETAILED DESCRIPTION OF THE EXEMPLARY EMBODIMENTS

### Overview and Terminology

Hereinafter, preferred embodiments of a wearable device for a pet of the present disclosure will be described with reference to the accompanying drawings. Like reference numerals in each figure indicate like members. In addition, specific structural or functional descriptions of the embodiments of the present disclosure are provided only for the purpose of describing the embodiments according to the present disclosure. Unless otherwise defined, all terms used herein including technological or scientific terms have the same meanings as those generally understood by a person with ordinary skill in the art. Terms defined in generally used dictionaries shall be construed that they have meanings matching those in the context of a related art, and shall not be construed in ideal or excessively formal meanings unless they are clearly defined in the present application.

The present disclosure seeks to provide a solution that is worn on body parts of a pet to collect detection information including biometric information, movement information, surrounding environment information, or a combination thereof, from not only the pet but also surroundings of the pet, and registers the collected detection information with a pet management server, so that an authorized user (including family members) may access the pet management server anytime and anywhere freely and view the detection information for the pet to extensively monitor health status and surroundings of the pet.

The present disclosure has been made keeping in mind the above problems occurring in the related art, and the present disclosure is intended to provide a wearable device for a pet that is worn on body parts of the pet to collect detection information including biometric information (body temperature, pulse, etc.), movement information (moving direction, speed, etc.), surrounding environment information (sound, ambient temperature, etc.), or a combination thereof, from not only the pet but also surroundings of the pet, and to register the collected detection information with a pet management server.

An objective of the present disclosure is to provide a wearable device for a pet that receives detection information generated when a pet uses any of the peripheral devices provided in a fixed or flexible form in a predetermined space such as a veterinary hospital, dog cafe, home, office, etc. from the corresponding peripheral device through near field communication (NFC), Bluetooth Low Energy (NFC), Bluetooth, etc. and registers the detection information with the pet management server.

An objective of the present disclosure is to provide a wearable device for a pet that enables direct transmission of detection information to a user terminal through NFC, NFC, Bluetooth, etc. if the user is located in close proximity to their pet.

An objective of the present disclosure is to provide a wearable device for a pet that uses multiple sensors to increase measurement accuracy of pet biometrics when measuring pet biometrics, determines the average or median value of measured values of individual sensors as the final measured value for the target pet, and allows correction of measurements by reflecting external factors, namely weather changes.

An objective of the present disclosure is to provide a wearable device for a pet that generates vibration or sound by operation of a user to draw and divert attention of their pet or make the pet move.

An objective of the present disclosure is to provide a wearable device for a pet that tracks movements of a pet by checking the location of a pet through a GPS receiver and registering the checked location information with the pet management server in order to keep track of an exercise record of the pet or respond to situations in which the pet is lost.

An objective of the present disclosure is to provide a wearable device for a pet that transmits the detection information to the pet management server for registration through low-power wide-area networks including long range (LoRa), long term evolution category (LTE cat), WiFi or narrowband Internet of Things (NB-IoT) via repeaters including base stations or access points.

An objective of the present disclosure is to provide a wearable device for a pet that transmits the detection information to the pet management server for registration by using a non-subscribed user terminal that is not subscribed to a paid communication service or a subscribed user terminal that is subscribed to a paid communication service as a repeater.

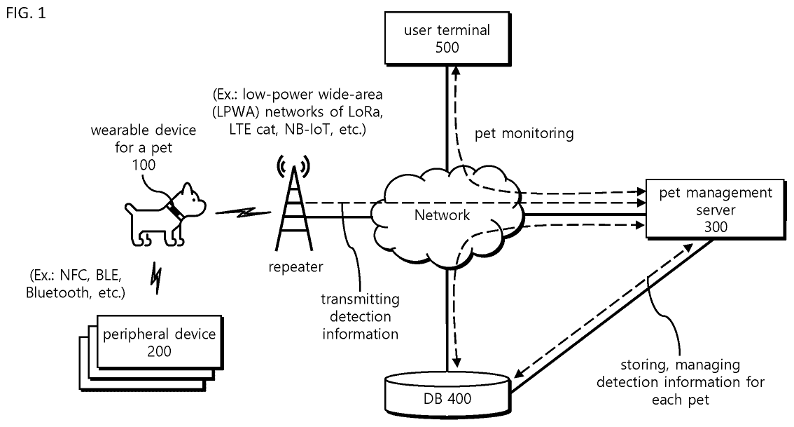

FIG. 1 is a view showing the overall configuration for the processing of detection information related to the health status and behavior of a pet, including a wearable device for a pet according to an embodiment of the present disclosure.

As shown in FIG. 1, a wearable device for a pet 100 according to an embodiment of the present disclosure may be manufactured in various shapes that can be worn on body parts such as the neck, ears, legs, and torso of pets including dogs and cats.

The wearable device for a pet 100 includes various sensors such as a biometric sensor, a motion sensor, a temperature sensor, and a sound sensor therein. As such, the wearable device for a pet 100 detects biometric information such as body temperature or pulse and movement information such as moving direction or speed of a pet active indoors or outdoors, as well as surrounding environmental information such as sound or ambient temperature, and transmits detection information including biometric information, movement information, surrounding environment information, or a combination thereof to a pet management server 300 through a network for registration.

At this time, the wearable device for a pet 100 transmits the detection information to the pet management server 300 for registration and management through low-power wide-area networks including long range (LoRa), long term evolution category (LTE cat), WiFi or narrowband Internet of Things (NB-IoT) via repeaters including base stations or access points.

Accordingly, a user (including family members) who manages the pet may access the pet management server 300 through a user terminal 500 possessed by the user, and check the behavior or status of the pet based on the registered detection information.

In addition, the wearable device for a pet 100 needs to provide detection information generated when the pet uses any of the peripheral devices 200 provided in a fixed or flexible form in a predetermined space such as a veterinary hospital, dog cafe, home, office, etc. to the pet management server 300 to be registered.

However, in most cases, the corresponding peripheral device 200 may not be able to perform a direct communication connection with the pet management server 300 to transmit the detection information as for which pet has used the corresponding peripheral device 200. Thus, the wearable device for a pet 100 may receive usage information (i.e., biometric information, movement information, and surrounding environment information, such as moving distance in the case of toys, photographed images or inspection data in the case of medical devices, etc.) through near field communication (NFC), Bluetooth low energy (BLE), Bluetooth, etc., and transmit the received information, detected according to the use of the peripheral device 200, together with the detection information directly measured from the pet to the pet management server 300 to be registered.

This is one of the features of the present disclosure. When the pet uses any one peripheral device in a situation where there are many peripheral devices, the wearable device for a pet 100 collects information on which peripheral device the pet uses and how it is used from that peripheral device and provides the collected information to the pet management server 300 to be registered. In this way, by simultaneously utilizing the information collected from peripheral devices as well as the detection information collected by itself, it is possible to expand the area in which behavior or condition of the pet may be monitored to various play equipment or medical devices, enabling more extensive and detailed monitoring.

Meanwhile, when the user is located in close proximity to the pet, the wearable device for a pet 100 may directly transmit detection information to the user terminal possessed by the corresponding user through NFC, BLE, Bluetooth, etc.

The peripheral device 200 is a tool or device of various shapes and types in a fixed or flexible form, which is provided in a home or office, as well as a veterinary hospital or dog cafe, etc., that can be used by the pet. The peripheral device 200 includes a pet house, tableware, toys, medical measuring devices and instruments, medical equipment, and the like.

Similar to the wearable device for a pet 100, the peripheral device 200 may be provided with various sensors such as biometric sensor, acceleration sensor, and gyro sensor, as well as communication module supporting NFC, BLE or Bluetooth for communication connection with the wearable device for a pet 100, and transmits various types of medical information obtained by examining the pet together with the information detected as the pet uses the peripheral device 200 including biometric information, movement information, surrounding environment information to the wearable device for a pet 100.

At this time, the pet management server 300 receives detection information including biometric information, movement information, surrounding environment information, or a combination thereof measured directly from the pet by the wearable device for a pet 100 via repeaters including base stations or access points connected to low-power wide-area networks. Then, the pet management server 300 classifies the received detection information by pet, stores the classified detection information in the database 400, and manages the stored detection information.

In addition, in the process of storing and managing the detection information for each pet in the database 400, the pet management server 300 provides the detection information of the pet to the user terminal 500 at the request of the user terminal 500 that has performed communication access through broadband wired/wireless networks such as 5G, WiFi, wired/wireless Ethernet, CDMA, LTE, etc., which are networks different from those used by the wearable device for a pet 100, so that behavior or condition of the pet may be checked.

At this time, the reason the pet management server 300 uses different and separate networks (or performs a communication connection through different sessions) in the process of communication connection and transmission/reception of the detection information with the wearable device for a pet 100 or the user terminal 500 is to enable the use of low-power wide-area networks available at low communication costs when receiving and registering detection information of each pet from the wearable device for a pet 100 without having to use broadband wired/wireless networks with high communication costs based on the fact that no urgent problem arises even if the user who manages various pets does not immediately check the behavior or condition of the pet in non-real time.

In the database 400, detection information including biometric information, movement information, surrounding environment information for each pet is stored and managed in a time-sequential manner.

The user terminal 500 is a communication terminal such as a smartphone or tablet owned by the user who is the owner of the pet. The user terminal 500 performs communication access to the pet management server 300 on web-based or through push/pull, and receives the detection information stored in the database 400 from the pet management server 300 to check the behavior or condition of the pet.

At this time, the detection information is information provided by the wearable device for a pet 100 prior to the current time and accumulated and registered in time series. Users may check the activity level and health status of their pet that is at home or other places through the detection information.

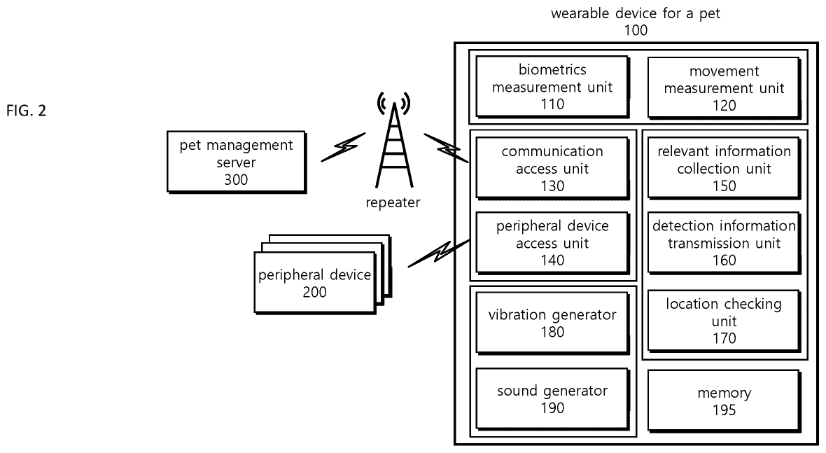

FIG. 2 is a block diagram showing the configuration of the wearable device for a pet according to the embodiment of the present disclosure in more detail.

As shown in FIG. 2, the wearable device for a pet 100 includes a biometrics measurement unit 110, a movement measurement unit 120, a communication access unit 130, a peripheral device access unit 140, a relevant information collection unit 150, a detection information transmission unit 160, a location checking unit 170, a vibration generator 180, a sound generator 190, and a memory 195.

The biometrics measurement unit 110 and the movement measurement unit 120 are measuring part of the wearable device for a pet 100. The communication access unit 130 and the peripheral access unit 140 take roles of connecting different devices via communication link in the wearable device for a pet 100. The vibration generator 180 and the sound generator 190 take roles of generating vibration and sound as devices. The relevant information collection unit 150, the detection information transmission unit 160, the location checking unit 170 are for information processing parts and may be implemented on a processor in the wearable device for a pet 100.

The biometrics measurement unit 110 includes a biometric sensor that measures biometrics such as body temperature or pulse rate of the pet by directly touching a body part of the pet or in a non-contact manner. The biometrics measurement unit 110 outputs the measured biometrics of the pet to the detection information transmission unit 160.

The biometric sensor may include a plurality of infrared sensors. For example, assuming that body temperature of the pet is measured by installing an infrared sensor from the lower end of the wearable device for a pet 100 worn on the neck of the pet toward the chin of the pet, depending on the distance between the neck and chin of the pet, and the sensitivity of the infrared sensor, body temperature measurement may be inaccurate. That is, to improve measurement accuracy when measuring the body temperature of the pet, in the present disclosure, the plurality of infrared sensors are provided in the wearable device for a pet 100, and an average value or a median value of the measured values measured by individual infrared sensors may be determined as the final body temperature measurement value of the pet.

In addition, in the process of measuring the body temperature of the pet, the wearable device for a pet 100 may correct the determined body temperature of the pet according to changes in external temperature or weather. For example, establish a table in advance that shows the relationship between the external temperature and weather, the body temperature of the pet measured by the sensor, and the corrected body temperature, and then correct the body temperature of the pet according to changes in external temperature or weather using the built table. It is also possible to use artificial intelligence that extracts the final body temperature value by inputting the measured value of the current temperature of the pet and information about the external temperature or weather.

The movement measurement unit 120 includes an accelerometer and a gyro sensor for measuring the movement or moving direction of the pet, and outputs the measured movement information of the pet to the detection information transmission unit 160.

In addition, although not shown in FIG. 2, the wearable device for a pet 100 may include a temperature sensor to measure the temperature around the pet and a sound sensor to detect sound.

The communication access unit 130 performs a communication connection with the pet management server 300, and transmits detection information measured by the biometrics measurement unit 110 and the movement measurement unit 120 and confirmed by the detection information transmission unit 160 or the detection information and relevant information generated as the pet uses the peripheral device (i.e., detection information measured by the peripheral device) collected by the relevant information collection unit 150 to the pet management server 300.

That is, the communication access unit 130 transmits the detection information to the pet management server 300 via repeaters including base stations or access points connected to the low-power wide-area networks including LoRa, LTE cat, WiFi or NB-IoT.

At this time, the relevant information is detection information measured by the peripheral device 200 as the pet approaches or uses the peripheral device 200, and includes biometrics of the pet, movement information detected by the peripheral device 200, and environment information around the peripheral device 200.

In addition, the communication access unit 130 receives an operation control signal provided from the pet management server 300 and provides the received signal to the vibration generator 180 or the sound generator 190,

The peripheral device access unit 140 communicates with various peripheral devices 200 including pet toys, play equipment, feeding trays, tableware, medical measuring devices and instruments, and medical equipment located or installed around the pet at home, office, dog cafe, etc. through NFC, BLE, Bluetooth, etc.

In addition, the peripheral device access unit 140 receives relevant information (i.e., detection information) including biometric information, movement information, surrounding environment information detected by the peripheral device 200 generated as the pet uses the peripheral device 200. For example, information on biometrics detected by the peripheral device 200 may be body temperature information of the pet measured by a temperature sensor provided in a toy when the pet approaches or bites the toy.

The relevant information collection unit 150 collects relevant information related to the pet provided from the peripheral device 200 through the peripheral device access unit 140, and provides the collected relevant information to the detection information transmission unit 160.

That is, the relevant information collection unit 150 performs a function of collecting and expanding related information including biometric information, movement information, surrounding environment information of the pet, or a combination thereof measured by the peripheral device 200 as the pet approaches or uses the peripheral device 200.

The detection information transmission unit 160 transmits detection information measured by the biometrics measurement unit 110 and the movement measurement unit 120 to the pet management server 300 through the communication access unit 130.

In addition, when the peripheral device access unit 140 and the relevant information collection unit 150 collect relevant information including biometric information, movement information, surrounding environment information generated as the pet uses the peripheral device 200 from the peripheral device 200, the detection information transmission unit 160 combines the relevant information provided by the peripheral device 200 with the detection information measured by the biometrics measurement unit 110 and the movement measurement unit 120, and transmits the combined information to the pet management server 300.

The location checking unit 170, which is a GPS receiver, checks the location information of the pet and provides the checked location information to the detection information transmission unit 160, so that the location information of the pet is transmitted to the pet management server 300 to be registered.

For example, by checking the location of the pet through the GPS receiver and registering the location information of the checked pet in the pet management server 300, movements of the pet may be continuously tracked and thus the amount of exercise done by the pet may be checked. Moreover, in case the user loses their pet, by checking the location where the pet is currently located through the stored location information of the pet, the user is able to find their pet.

The vibration generator 180 generates vibration according to a control signal received from the pet management server 300 through the communication access unit 130.

The sound generator 190 outputs a preset sound or a recorded voice of a user through a speaker according to a control signal received from the pet management server 300 through the communication access unit 130.

That is, the vibration generator 180 and the sound generator 190 performs a function of generating vibration or sound to draw and divert attention of a pet or make the pet move by the operation of a user located remotely. The reason for generating such vibration or sound is to attract the attention of the pet or make the pet move when the user thinks there is no movement of the pet while monitoring their pet from a distance, or to prevent the pet from overreacting to other pets or people, or to locate the pet.

The memory 195 stores various operation programs used in the wearable device for a pet 100. The memory 195 performs a function of temporarily storing detection information measured by the biometrics measurement unit 110 and the movement measurement unit 120 or relevant information collected from the peripheral device 200 through the peripheral device access unit 140 and the relevant information collection unit 150.

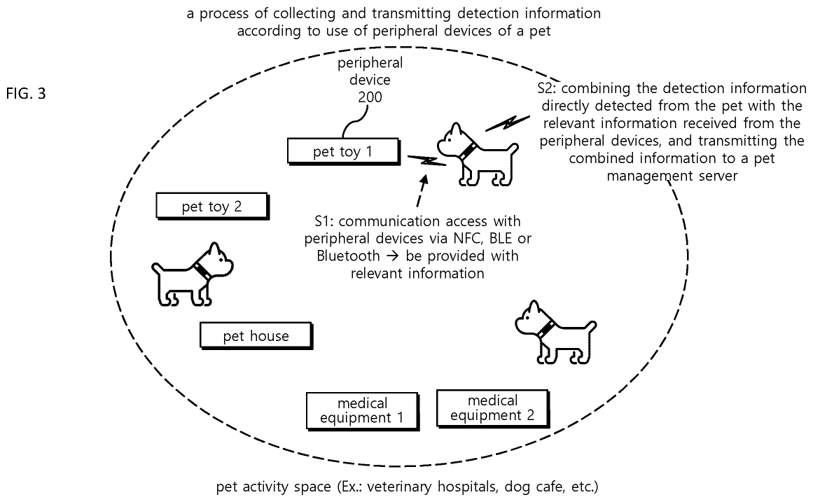

FIG. 3 is a view showing the process of transmitting the detection information received from peripheral devices to the pet management server together with the detection information directly detected from the pet in the wearable device for a pet according to the embodiment of the present disclosure.

As shown in FIG. 3, in houses, which is the activity space where pets are mainly located, as well as in certain spaces such as veterinary hospitals and dog cafes, various shapes and types of peripheral devices 200 such as pet houses, tableware, toys, medical measuring devices and instruments, and medical equipment may be fixedly installed or provided in a flexible form. That is, peripheral devices 200 such as a pet toy 1 and 2, and medical equipment 1 and 2 may be placed in the pet activity space.

In this case, the wearable device for a pet 100 needs to collect detection information generated as the pet uses the peripheral device 200, and register the collected information in the pet management server 300.

For example, assuming a pet plays with the pet toy 1 as shown in FIG. 3, detection information about the distance moved or moved by the pet is measured in the pet toy 1.

Then, the wearable device for a pet 100 is provided with the detection information from the pet toy 1 through NFC, BLE, Bluetooth, etc. (S1)

Next, the wearable device for a pet 100 combines the detection information received from toy 1 with information directly detected from the pet, and transmits the combined information to the pet management server 300 via a repeater (S2). At this time, the detection information generated from the peripheral device 200 is classified and stored as the detection information for the corresponding pet by the wearable device for a pet 100 worn on the corresponding pet.

Accordingly, users are able to perform more extensive and diverse monitoring on the basis of the detection information generated as the pet uses the peripheral device 200 on top of the information directly measured from the pet. That is, it is possible to broadly expand the monitoring area to a wide range including pet toys, various audio and video equipment, and sensors.

Meanwhile, as shown in FIGS. 1 to 3, that the wearable device for a pet 100 transmitting the detection information (i.e., information directly measured from the pet and information provided by the peripheral device) to the pet management server 300 through low-power wide-area networks is described as an example.

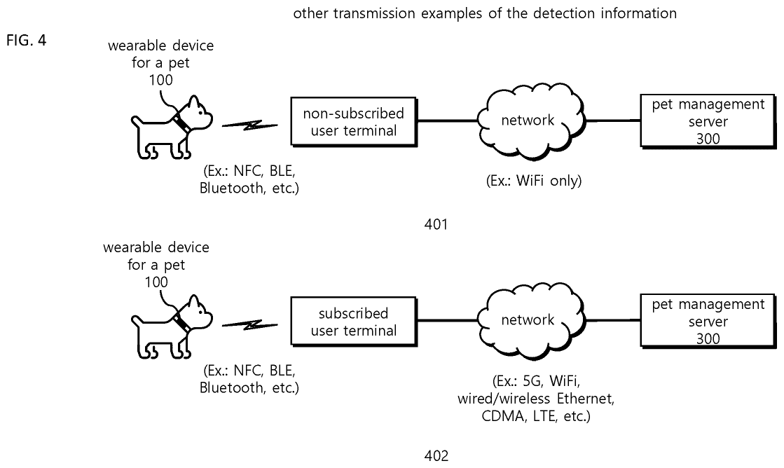

However, the present disclosure is not limited thereto, and the wearable device for a pet 100 may use a different transmission system when transmitting the detection information to the pet management server 300, which will be described with reference to FIG. 4 as follows.

FIG. 4 shows views of other transmission examples 401 and 402 of the detection information used in the wearable device for a pet according to the embodiment of the present disclosure.

As shown in 401 and 402 of FIG. 4, the wearable device for a pet 100 may transmit the detection information to the pet management server 300 for registration by using a non-subscribed user terminal that is not subscribed to a paid communication service or a subscribed user terminal that is subscribed to a paid communication service as a repeater.

For example, as shown by 401 in FIG. 4, the wearable device for a pet 100 may transmit the detection information to the non-subscribed user terminal through NFC, BLE, Bluetooth, etc., and then the non-subscribed user terminal may transmit the detection information received from the wearable device for a pet 100 to the pet management server 300 through WiFi.

That is, the non-subscribed user terminal is a smartphone that can only use WiFi. When the detection information of the pet is received from the wearable device for a pet 100 while the pre-installed application program is running, the non-subscribed user terminal transmits the detection information to the pet management server 300 through WiFi. In this way, using the non-subscribed user terminal has the advantage that no additional cost is required.

In addition, as shown by 402 in FIG. 4, when the user terminal 500 possessed by the user is located near the wearable device for a pet 100, the wearable device for a pet 100 transmits the detection information to the user terminal 500 through NFC, BLE, Bluetooth, etc., and then the user terminal 500 may transmit the detection information received from the wearable device for a pet 100 to the pet management server 300 through broadband wired/wireless networks such as 5G, WiFi, wired/wireless Ethernet, CDMA, LTE, etc. At this time, the user terminal 500 is a subscribed user terminal that is subscribed to a paid communication service, and a user terminal possessed by the owner of the pet as well as user terminals possessed by other family members may be used.

That is, the detection information received from the wearable device for a pet 100 is transmitted to the pet management server 300 through communication networks (5G, LTE, WiFi, etc.) used by the user terminal 500 while a hidden type of application program is running in the user terminal 500 located around the wearable device for a pet 100. In this process, the user terminal 500 may be set as not to directly check the detection information received from the wearable device for a pet 100.

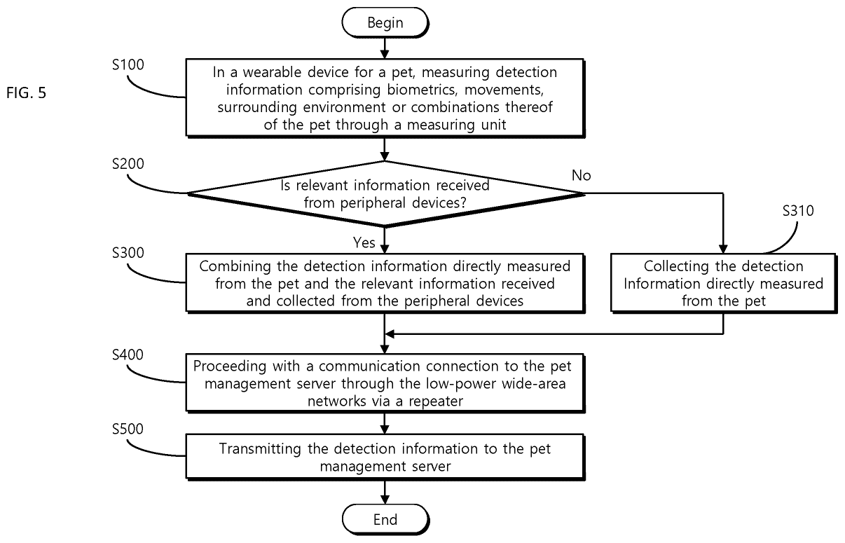

Next, an embodiment of a method for operating the wearable device for a pet according to the present disclosure configured as described above will be described in detail with reference to FIG. 5. At this time, the order of each step according to the method of the present disclosure may be changed by a user environment or a person skilled in the art.

FIG. 5 is a flowchart showing in detail the operation process of a method for operating the wearable device for a pet according to the embodiment of the present disclosure.

As shown in FIG. 5, the wearable device for a pet 100 preforms measuring (S100) to measure detection information including information on the biometrics, movements, surrounding environment of the pet, or a combination thereof using various sensors such as a biometric sensor, an accelerometer, a gyro sensor, and a temperature sensor therein.

In addition, the wearable device for a pet 100 determines (S200) whether the peripheral devices 200 including a pet house, tableware, toys, medical measuring devices and instruments, medical equipment, and the like located around the pet in a fixed or flexible form are connected for communication, and relevant information including information on biometrics, movements, surrounding environment detected by the peripheral devices 200 generated as the pet uses the peripheral devices 200 (i.e., detection information measured by the peripheral device) is received.

That is, the wearable device for a pet 100 determines whether the relevant information is received from the peripheral devices 200 located around the pet through NFC, BLE, Bluetooth, etc.

As a result of determination in step S200, when the relevant information generated as the pet uses the peripheral devices 200 is received from the peripheral devices 200, the wearable device for a pet 100 combines (S300) the detection information measured using various sensors provided therein in step S100 and the relevant information received and collected from the peripheral devices 200.

On the contrary, as a result of determination in step S200, when the relevant information generated as the pet uses the peripheral devices 200 is not received from the peripheral devices 200, the wearable device for a pet 100 collects (S310) the detection information measured from the pet.

Next, the wearable device for a pet 100 proceeds (S400) with a communication connection to the pet management server 300 through the low-power wide-area networks including LoRa, LTE cat, WiFi or NB-IoT via repeaters including base stations or access points.

At this time, as previously described, the wearable device for a pet 100 may proceed with a communication connection to the pet management server 300 by using a non-subscribed user terminal that is not subscribed to a paid communication service or a subscribed user terminal that is subscribed to a paid communication service as a repeater, in addition to a method of performing communication connection to the pet management server 300 through a repeater connected to the low-power wide-area networks.

After performing the communication connection to the pet management server 300 in step S400, the wearable device for a pet 100 transmits(S500) the combined detection information combining the detection information directly measured from the pet with the detection information received from the peripheral devices 200 in step S300, or the detection information directly measured from the pet collected in step S310 to the pet management server 300.

Accordingly, the pet management server 300 registers and stores the detection information provided by the wearable device for a pet 100 in the database 400, and later, when the user accesses the pet management server 300 through his/her user terminal 500, the pet management server 300 provides the detection information of the pet so that the user may check the behavior or condition of their pet.

Meanwhile, although not shown in FIG. 5, the wearable device for a pet 100 may further perform a location checking step of checking location information of the pet. That is, by transmitting and registering the current location information of the pet to the pet management server 300, it is possible to track a movement path of the pet to check an exercise record of the pet, or to respond to pet loss situation.

In addition, the wearable device for a pet 100 may generate vibration or sound under the control of the pet management server 300. That is, by generating vibration or sound, it is possible to attract attention of a pet and make the pet move when the pet is not moving, or to prevent the pet from overreacting to other pets or people.

As such, the present disclosure receives detection information periodically measured directly from the target pet including biometric information, movement information, surrounding environment information, or a combination thereof, as well as detection information generated when the pet uses any of the peripheral devices provided in a fixed or flexible form in spaces such as a veterinary hospital, dog cafe, home, office, etc. from the corresponding peripheral device through NFC, BLE, Bluetooth, etc., and registers the detection information win the pet management server, so that the user who is the owner of the pet (including family members) can freely access the pet management server anytime, anywhere and browse the time-sequentially stored information, enabling extensive monitoring of health status and surroundings of the pet.

Thus far, the present disclosure has been described with reference to the embodiments shown in the drawings. However, this is merely illustrative, and those skilled in the art to which the present disclosure pertains may easily understand that various modifications and equivalent other embodiments are possible from the embodiments of the present disclosure. Therefore, the technical protection scope of the present disclosure should be defined only by the appended claims.

What is claimed is:

1. A wearable device for a pet comprising:
a biometrics measurement unit configured to measure biometrics of the pet;
a movement measurement unit configured to measure movements of the pet; and
a relevant information collection unit configured to collect relevant information related to the pet from peripheral devices near the pet,
wherein the relevant information collection unit is configured to expand and collect the relevant information including biometric information, movement information, surrounding environment information, or a combination thereof generated as the pet approaches or uses the peripheral devices.

2. The wearable device of claim 1, further comprising:
a detection information transmission unit configured to transmit the measured and collected detection information including biometric information, movement information, and collection information, or a combination thereof to a pet management server, and
wherein the wearable device transmits the detection information to the pet management server for registration through low-power wide-area networks including LoRa, LTE cat, WiFi or NB-IoT via a repeater including a base station or an access point, so that the wearable device enables a user to connect to the pet management server with a terminal of the user and to monitor a behavior or a condition of the pet.

3. The wearable device of claim 1, further comprising:
a peripheral device connection unit configured to perform a communication connection with peripheral devices located around the pet and receive the detection information including biometric information, movement information, or surrounding environment information, detected by the peripheral devices,
wherein the peripheral device connection unit receives the detection information, detected by the peripheral devices from the peripheral devices through NFC, BLE, or Bluetooth, and
wherein the peripheral devices comprise a pet house, tableware, toys, medical measuring devices and instruments, or a medical equipment.

4. The wearable device of claim 1, wherein the biometrics measurement unit is configured to measure the biometrics of the pet by using infrared sensors, and
the wearable device for the pet is configured to determine an average value or a median value of measurement values measured by each of the infrared sensors as the biometrics of the pet, and correct the biometrics of the pet by reflecting external temperature change.

5. The wearable device of claim 1, further comprising:
a location checking unit configured to check location information on the pet,
wherein the wearable device is configured to transmit the checked location information to the pet management server for registration, so that the wearable device enables the user to check movement amount of the pet or respond to a pet loss situation, by tracking a moving path of the pet in the pet management server.

6. The wearable device of claim 1, further comprising:
a vibration generator configured to generate vibration according to a control signal received from the pet management server, and
a sound generator configured to output a preset sound or a recorded voice of a user according to a control signal received from the pet management server.

7. The wearable device of claim 1, wherein the wearable device is further configured to transmit the detection information including biometric information, movement information, and collection information, or a combination thereof to the pet management server, by using a non-subscribed user terminal that is not subscribed to a paid communication service or a subscribed user terminal that is subscribed to a paid communication service as a repeater,
wherein the non-subscribed user terminal is configured to receive the detection information from the wearable device for the pet through NFC, BLE, or Bluetooth, and transmit the received detection information to the pet management server only through WiFi,
while the subscribed user terminal is configured to receive the detection information from the wearable device for the pet through NFC, BLE, or Bluetooth, and transmit the received detection information to the pet management server through broadband wired or wireless networks such as 5G, WiFi, wired or wireless Ethernet, CDMA, or LTE.

8. A method for operating a wearable device for a pet, the method comprising:
measuring, by the wearable device for a pet, biometrics of a pet;
measuring, by the wearable device for a pet, movements of the pet; and
collecting relevant information related to the pet from peripheral devices near the pet,
wherein the collecting relevant information is configured to expand and collect the relevant information including biometric information, movement information, surrounding environment information, or a combination thereof generated as the pet approaches or uses the peripheral devices.

9. The method of claim 8, further comprising:
transmitting, by the wearable device for the pet, the measured and collected detection information to a pet management server,
wherein the method is further configured to transmit the detection information to the pet management server for registration through low-power wide-area networks including LoRa, LTE cat, WiFi or NB-IoT via a repeater including a base station or an access point, so that the method enables a user to connect to the pet management server with a terminal of the user and to monitor a behavior or a condition of the pet.

10. The method of claim 8, further comprising:
connecting, by the wearable device for the pet, peripheral devices near the pet to perform a communication connection with the peripheral devices located around the pet and receive the detection information including biometric information, movement information, or surrounding environment information, detected by the peripheral devices,
wherein the wearable device for the pat is configured to receive the detection information detected at the peripheral device through NFC, BLE, or Bluetooth from the peripheral devices,
wherein the peripheral devices comprise a pet house, tableware, toys, medical measuring devices and instruments, or a medical equipment.

11. The method of claim 8, wherein the measuring the biometrics includes measuring the biometrics of the pet by using infrared sensors, and
wherein the wearable device for the pet is configured to determine an average value or a median value of measurement values measured by each of the infrared sensors as the biometrics of the pet, and correct the biometrics of the pet by reflecting external temperature change.

12. The method of claim 8, further comprising:
checking, by the wearable device for the pet, location information on the pet, and
wherein the wearable device is configured to transmit the checked location information to the pet management server for registration, so that the wearable device enables the user to check movement amount of the pet or respond to a pet loss situation, by tracking a moving path of the pet in the pet management server.

13. The method of claim 8, further comprising:
generating vibration according to a control signal received from the pet management server, and
generating and outputting a preset sound or a recorded voice of a user according to a control signal received from the pet management server.

14. The method of claim 8, further comprising:
transmitting the detection information including biometric information, movement information, and collection information, or a combination thereof to the pet management server, by using a non-subscribed user terminal that is not subscribed to a paid communication service or a subscribed user terminal that is subscribed to a paid communication service as a repeater,
wherein the non-subscribed user terminal is configured to receive the detection information from the wearable device for the pet through NFC, BLE, or Bluetooth, and transmit the received detection information to the pet management server only through WiFi,
while the subscribed user terminal is configured to receive the detection information from the wearable device for the pet through NFC, BLE, or Bluetooth, and transmit the received detection information to the pet management server through broadband wired or wireless networks such as 5G, WiFi, wired or wireless Ethernet, CDMA, or LTE.
